(19) United States
(12) Patent Application Publication
(10) Pub. No.: US 2007/0287960 A1
(43) Pub. Date: Dec. 13, 2007

(54) Disposable infusion device with medicament level indicator

(75) Inventors: John M. Adams, Kirkland, WA (US); Clifton A. Alferness, Port Orchard, WA (US); Daniel Hawkins, Newcastle, WA (US)

Correspondence Address: Richard O. Gray, Jr.;GRAYBEAL JACKSON HALEY LLP, Suite 350, 155-108th Avenue NE, Bellevue, WA, 98004-5973, US

(73) Assignee: Seattle Medical Technologies

(21) Appl. No.: 11/801,989

(22) Filed: May 10, 2007

Related U.S. Application Data
(60) Provisional application No. 60/809,957, filed on May 31, 2006.

(51) Int. Cl. A61M 5/142

(52) U.S. Cl. 604/151

(57) ABSTRACT

A wearable infusion device delivers a liquid medicament, such as insulin, to a patient. The device comprises a base that contacts a patient's skin, a reservoir arranged to contain a liquid medicament to be delivered to beneath a patient's skin, a pump that causes the medicament to flow from the reservoir, and a level indicator that provides an indication of medicament level.

### CLAIM OF PRIORITY

This application claims priority to U.S. Provisional Application Ser. No. 60/809,957, filed on May 31, 2006, which is incorporated by reference.

### BACKGROUND OF THE INVENTION

Tight control over the delivery of insulin in both type I diabetes (usually juvenile onset) and type II diabetes (usually late adult onset), has been shown to improve the quality of life as well as the general health of these patients. Insulin delivery has been dominated by subcutaneous injections of both long acting insulin to cover the basal needs of the patient and by short acting insulin to compensate for meals and snacks. Recently, the development of electronic, external insulin infusion pumps has allowed the continuous infusion of fast acting insulin for the maintenance of the basal needs as well as the compensatory doses for meals and snacks. These infusion systems have shown to improve control of blood glucose levels, however, they suffer the drawbacks of size, cost, and complexity, which prevents many patients from accepting this technology over the standard subcutaneous injections. These pumps are electronically controlled and must be programmed to supply the desired amounts of basal and bolus insulin.

Hence, there is a need in the art for a simple, mechanically driven infusion device for both basal needs and boluses that is directly attached to the body and does not require any electronics to program the delivery rates. The insulin is preferably delivered through a small, thin-walled tubing (cannula) through the skin into the subcutaneous tissue similar to technologies in the prior art.

In the use of such a device, it would be most desirable if the user were able to discern a level of the liquid medicament, such as the amount remaining to be dispensed. However, providing such an indicator is difficult with prior art techniques given the required small size of the device and the desire for disposal of the device to be economically feasible. The present invention is directed to these and others issues concerning such a device.

### SUMMARY OF THE INVENTION

The invention provides a wearable infusion device comprising a base that contacts a patient's skin, a reservoir arranged to contain a liquid medicament to be delivered to beneath a patient's skin, a pump that causes the medicament to flow from the reservoir, and a level indicator that provides an indication of medicament level.

The indication of medicament level may be indicative of the volume of medicament remaining in the reservoir. The level indicator may include a display. The display may be an analog display. The analog display may include a mechanical indicator such as a display needle.

The reservoir may be formed of flexible material, to change shape responsive to the volume of medicament therein, and the level indicator may be responsive to the change in shape of the reservoir to indicate the medicament level. The device may further comprise a follower that follows the changes in shape of the reservoir and a linkage the couples the follower to the level indicator. The level indicator may include a mechanical indicator and the linkage may couple the follower to the mechanical indicator.

The device may further comprise a housing covering the base and enclosing the reservoir and the pump. The housing may include a window through which the level indicator is viewable. The level indicator may include a display needle.

### BRIEF DESCRIPTION OF THE DRAWINGS

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

FIG. 1 is a perspective view of an infusion device which may embody the present invention;

FIG. 2 is a perspective view of the device of FIG. 1 with its top cover removed and in a condition ready for having its reservoir filled with liquid medicament;

FIG. 3 is a perspective view of the infusion device of FIG. 1 with its top cover removed after its reservoir has been filled with liquid medicament;

FIG. 4 is perspective view with portions cut away of the infusion device of FIG. 1 illustrating the path of the liquid medicament within the device;

FIG. 5 is a top view of the device of FIG. 1 with its reservoir removed to illustrate the manner in which the liquid medicament is caused to flow within the device;

FIG. 6 is a perspective view of the infusion device of FIG. 1 with its reservoir removed illustrating further aspects of the pump thereof;

FIG. 7 is a perspective view of the infusion device of FIG. 1 with portions cut away to illustrate a safety check valve in its opened position;

FIG. 8 is a perspective view similar to FIG. 7 illustrating the safety check valve closed;

FIG. 9 is a perspective view of a reservoir which may be used in the infusion device of FIG. 1 in accordance with an alternate embodiment;

FIG. 10 is a perspective view with portions cut away of the reservoir of FIG. 9 illustrating further aspects thereof according to the invention;

FIG. 11 is a perspective view of another infusion device arranged in accordance with another embodiment of the present invention;

FIG. 12 is a sectioned perspective view of the device of FIG. 11 illustrating the reservoir and a linkage and display needle when the reservoir is empty; and,

FIG. 13 is a sectioned perspective view of the device of FIG. 11 illustrating the reservoir and a linkage and display needle when the reservoir is full.

### DETAILED DESCRIPTION OF THE INVENTION

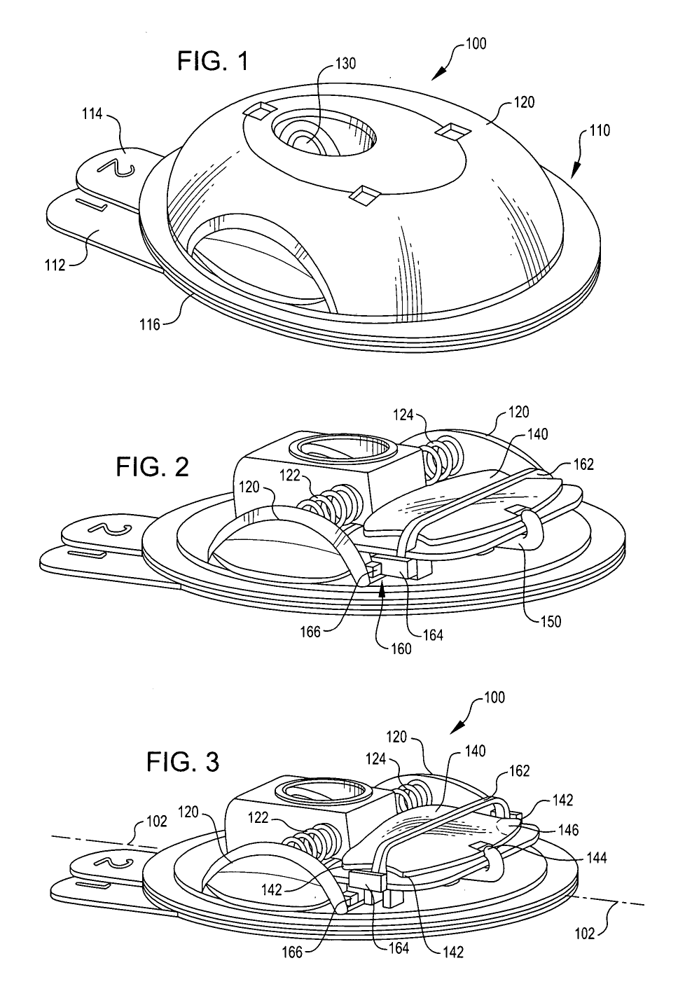

Referring now to FIG. 1, it illustrates an infusion device 100 embodying the present invention. The infusion device 100 may be useful, for example, in providing boluses of a liquid medicament, such as insulin, to be delivered beneath a patient's skin.

The device 100 generally includes a base 110, a top cover 120, and a cannula port 130. The base 110 prior to application to the patient's skin, carries a first tab member 112 and a second tab member 114. The first tab member 112, when removed, exposes a layer 116 of antiseptic material such as alcohol which may be rubbed against the skin of the patient in the area in which the device 100 is to be adhered. Once the antiseptic has been applied to the patient's skin, the second tab 114 is removed exposing an adhesive layer on the base 110 which is then used to adhere the device to the skin of the patient. Once the device is adhered to the skin of the patient, a cannula may be introduced into the device and beneath the skin of a patient through the cannula port 130.

As may be seen in FIG. 2, the device 100 further includes a pair of actuator push buttons 120 and a reservoir 140 arranged to contain the liquid medicament. As will be seen hereinafter, concurrent pressing of the actuator push buttons 120 causes the liquid medicament within the reservoir 140 to flow down a flexible conduit 150 and eventually beneath the skin of a patient. Each of the push buttons 120 are spring loaded by an associated spring 122 and 124 which return the push buttons 120 to their starting positions.

The reservoir 140 as shown in FIG. 2 does not yet contain the liquid medicament. A latch mechanism 160 precludes the push buttons 120 from being pressed when the reservoir 140 is empty. To that end, it will be noted that a follower bar 162 extends across the reservoir 140 and terminates at a latch member 164. A dog 166 is coupled to the push buttons 120 and engages the latch member 164 to preclude the actuator buttons 120 from being pushed when the reservoir is empty.

Referring now to FIG. 3, when the reservoir is filled as illustrated in FIG. 3, the follower bar 162 follows the expansion of the reservoir 140. To that end, the reservoir 140 is preferably formed of flexible material, such as plastic, and will expand upon being filled. The follower bar 162 follows the filling of the reservoir 140 to raise the latch member 164. When the reservoir is full, the latch member 164 is raised to such an extent that the dog 166 may pass thereunder to permit the push buttons 120 to be pressed to cause pumping of the liquid medicament to the patient. Again, the springs 122 and 124 assist in returning the push buttons 122 to a starting position.

The reservoir 140, as may be noted in FIG. 3, includes a plurality of raised portions 142 formed along its perimeter. This reservoir shape causes air pockets to be formed within the reservoir that traps air isolated from the reservoir outlet 144. Accordingly, the device 100 is intended to be worn with its major axis 102 horizontal. In such an orientation, air within the reservoir 140 may be trapped in the air pockets, such as air pocket 146.

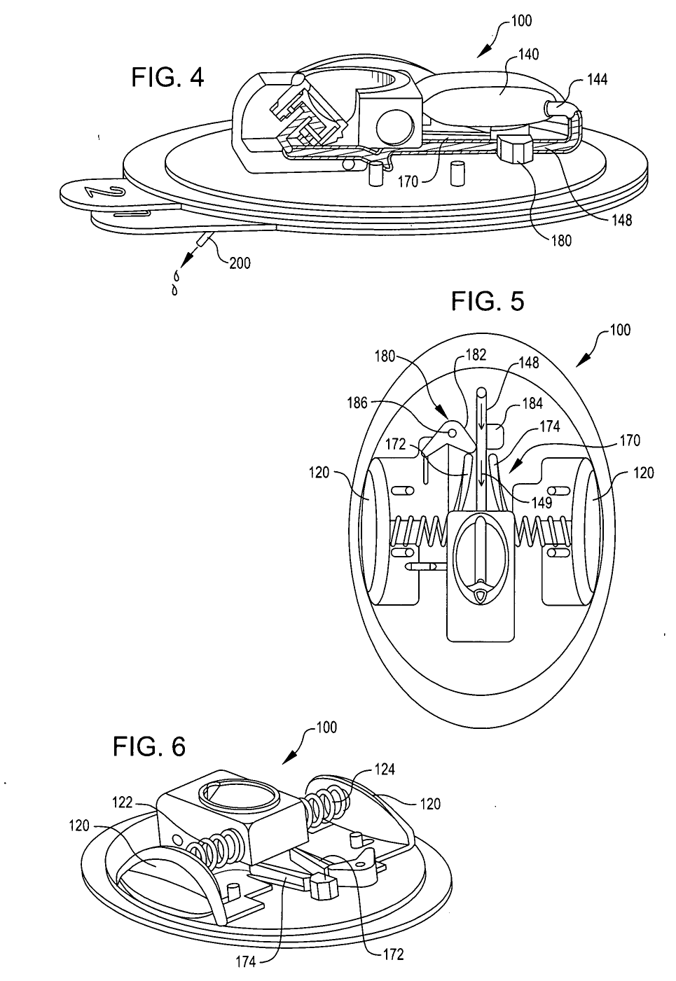

Referring now to FIG. 4, it illustrates the fluid flow path of the liquid medicament upon being pumped responsive to the pressing of the actuator buttons 120. The fluid flow path is shown in dashed lines in FIG. 4. As maybe noted, the fluid flow from reservoir 140 begins at the outlet 144 along a flexible conduit 148. The fluid medicament is propelled by a pump, such as a linear peristaltic pump 170 to be described hereinafter. It first flows through a valve 180 which may be provided to isolate the pump 170 from the reservoir 140 when the pump 170 pumps the fluid medicament. The valve 180, under some circumstances, is optional, as for example when a linear peristaltic pump of the type described herein is employed as will be fully described hereinafter.

The fluid continues to flow along the flexible conduit 148 to eventually arrive at the cannula 200. It is then delivered to the patient beneath the patient's skin.

FIGS. 5 and 6 show the peristaltic pump of the device 100 in greater detail. Here it may be seen that the peristaltic pump comprises a pair of pressure members 172 and 174. The pressure members 172 and 174 are disposed on opposite sides of the flexible conduit 148. The direction of fluid flow is indicated by the arrows 149 in a direction away from the reservoir (not shown). The pressure members 172 and 174 are spaced apart such that they become increasingly closer together in an upstream direction with respect to the fluid flow. Hence, when the pressure members 172 and 174 act upon the flexible conduit 148, they will serve to first pinch the flexible conduit closed and then, upon exerting additional pressure, squeeze the conduit to force the liquid medicament in the downstream direction.

As previously mentioned, the valve 180 is optional. If the pump utilized is not a pump as illustrated herein that first closes off the conduit, the valve 180 may be coupled to the actuator buttons 120 so that the valve 180 closes the conduit 148 before pressure is exerted on the flexible conduit 148 by the pump. To that end, the valve 180 includes a first valve member 182 and a second stationary valve member 184. Valve member 182 pivots about pivot point 186 upon the pressing of the actuator buttons 120 to pinch the flexible conduit closed against the stationary member 184.

In FIG. 6, it may be more clearly seen that each pressure member 172 and 174 is integrally formed with an associated one of the actuator buttons 120. More specifically, each pressure member may be formed as one piece with its actuator button 120. Because the device 100 is intended to be disposable, the actuator buttons and hence the pressure members 172 and 174 may be formed of plastic.

Once the actuator buttons 120 are pressed and the peristaltic pump 170 causes the liquid medicament to flow down the flexible conduit 148, the actuator buttons 120 are returned to their starting positions by their respective springs 122 and 124. At this point in time, the flexible conduit 148 is charged with fluid to cause the fluid medicament to exit the cannula 190 as illustrated in FIG. 4. To guard against back pressure within the cannula 190 and flexible conduit which would otherwise lessen the amount of liquid medicament received by the patient, a check valve 190 is provided. The check valve 190 is downstream from the pump 170 and performs at least two functions. Firstly, the check valve 190 when closed precludes back flow of the medicament and assures that the medicament within the flexible conduit from the check valve to the cannula 200 is eventually diffused into the patient. It also precludes unintended leaking of the liquid medicament into the patient in between actuations of the push buttons 120.

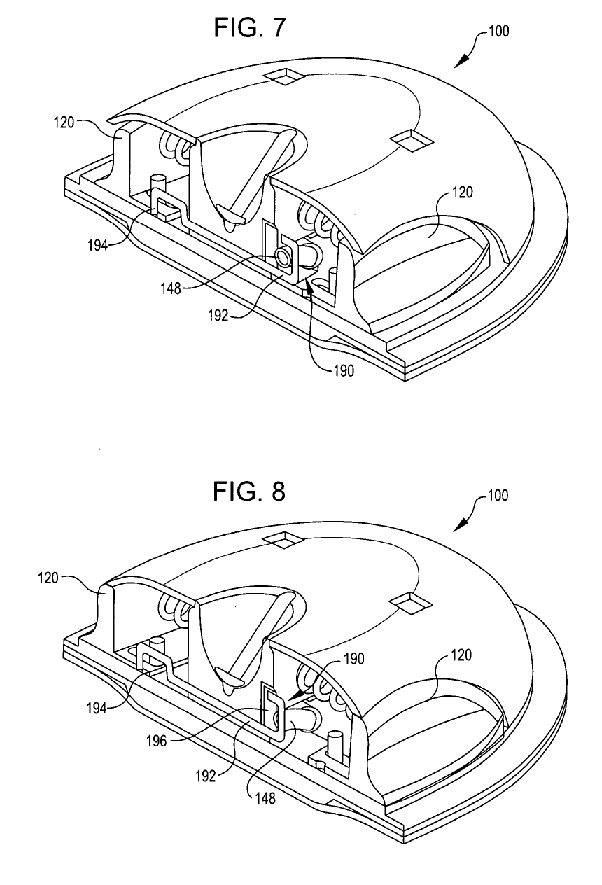

With particular reference to FIGS. 7 and 8, in FIG. 7, it will be noted that the valve 190 is formed by a closing member 192 which is coupled to an actuator push button 120 at an attachment point 194. When the actuator buttons 120 are concurrently pressed, the closure member 192 slides to the position indicated in FIG. 7 to an opened position to permit fluid flow through the flexible conduit 148. As may be seen in FIG. 8, when the actuator buttons 120 are released, the closure member 192 is caused to move in a direction towards the flexible conduit 148 and eventually pinches the flexible conduit 148 closed between the closure member 192 and a stationary wall 196. Once the valve 190 is closed as shown in FIG. 8, liquid medicament will not be permitted to inadvertently drip from the reservoir, flow through the conduit, and be delivered to the patient.

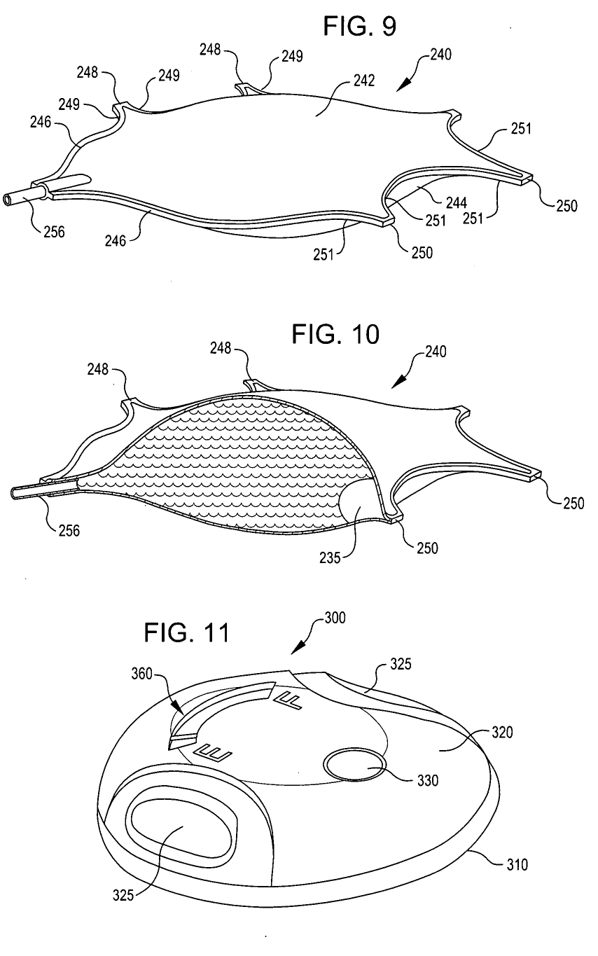

Referring now to FIGS. 9 and 10, they show an alternative flexible reservoir which may be used in the infusion pump according to the invention. The flexible reservoir 240 is formed of flexible sheet material including a sheet 242 and a sheet 244. The sheet materials 242 and 244 are sealed along a peripheral seal line 246. As may be clearly noted in FIGS. 9 and 10, the reservoir 240 is shaped to form raised portions 248 on one side of the reservoir and raised portions 250 on the opposite side of the reservoir. The raised portions 248 and 250 may be pointed regions having concave sidewalls. For example, pointed regions 248 have concave sidewalls 249 and pointed regions 250 have concave sidewalls 251.

When the reservoir 240 is deployed in an infusion device, such as infusion device 100 of FIG. 1, it may be disposed so that the raised regions 250 and 248 are along a pane having a substantially vertical component. With the reservoir 240 being disposed such that the raised regions 250 are above the raised regions 248, air pockets, such as air pocket 253 will be formed within the reservoir 240. The air pocket 253 is isolated from the outlet 256 to assure that no air will become entrapped in the liquid medicament being delivered to the patient.

Referring now to FIG. 11, it illustrates another infusion device 300 embodying the present invention. Like the infusion device 100, the infusion device 300 may be useful in providing boluses of a liquid medicament, such as insulin, to beneath a patient's skin.

The device 300 generally includes a base 310, a top cover or housing 320, and a cannula port 330. The base 310 is shown ready to be adhered to a patient's skin. Prior to application to the patient's skin, the device may carry first and second tab members as previously described in connection with the device 100. Once the device is adhered to the skin of the patient, a cannula (not shown) may be introduced into the device and beneath the skin of a patient through the cannula port 330.

The device 300 further includes a pair of actuator push buttons 325 and a reservoir 340 (FIG. 12) arranged to contain the liquid medicament. As in previous embodiments, concurrent pressing of the actuator push buttons 325 causes the liquid medicament within the reservoir 340 to flow down a flexible conduit (not shown) and eventually beneath the skin of a patient. To that end, the infusion device 300 may include a pump (not shown) of the type as previously described. Each of the push buttons 325 is preferably spring loaded to return the push buttons 325 to their starting positions.

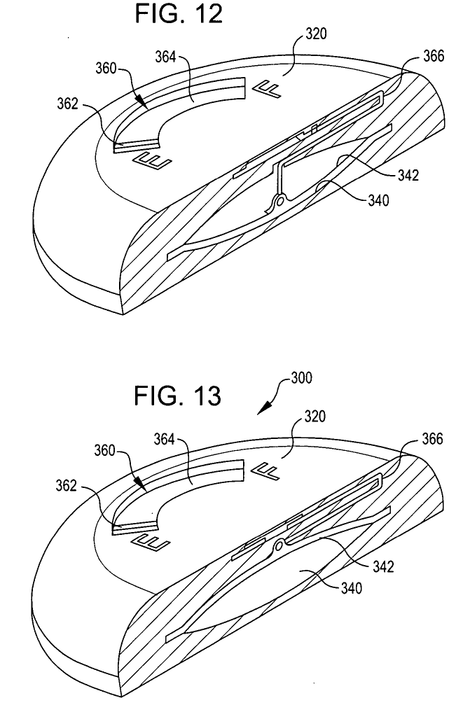

The reservoir 340 as shown in FIG. 12 does not yet contain the liquid medicament. This is accurately indicated on a level indicator 360. The level indicator includes a mechanical display needle 362 that is pivotably mounted and viewable through a window 364 of the housing 320. The display needle 362 is coupled to the reservoir 340 by a linkage 366. The reservoir includes a flexible membrane 342 that changes shape as the reservoir is filled and emptied. When the reservoir 340 is filled as illustrated in FIG. 13, the reservoir 340 expands and the flexible membrane 342 is deflected upward in the drawing. This causes the linkage 366 to push the display needle 362 about its pivot (not shown) so that it indicates that the reservoir 340 is full. In this same manner, but in the reverse direction, the level indicator 360 will indicate the level of remaining liquid medicament as the reservoir 340 contracts as it is emptied.

Hence, as may be seen from the foregoing, the present invention provides a simple, mechanically driven infusion device that provides boluses of liquid medicament, such as insulin, and which may directly attached to the body of a patient. The device does not require any electronics to deliver or program the delivery of the medicament. The liquid medicament, such as insulin, may be delivered through a small cannula into the subcutaneous tissue of the patient as is common in the art. The device further includes a level indicator that indicates medicament level. The level indicator provides accurate indications of medicament level and adds little cost to the device, maintaining its disposability.

While particular embodiments of the present invention have been shown and described, modifications may be made, and it is therefore intended in the appended claims to cover all such changes and modifications which fall within the true spirit and scope of the invention as defined by those claims.

What is claimed is:

1. A wearable infusion device comprising:
a base that contacts a patient's skin;
a reservoir arranged to contain a liquid medicament to be delivered to beneath a patient's skin;
a pump that causes the medicament to flow from the reservoir; and
a level indicator that provides an indication of medicament level.

2. The device of claim 1, wherein the indication of medicament level is indicative of the volume of medicament remaining in the reservoir.

3. The device of claim 1, wherein the level indicator includes a display.

4. The device of claim 3, wherein the display is an analog display.

5. The device of claim 4, wherein the analog display includes a mechanical indicator.

6. The device of claim 5, wherein the mechanical indicator comprises a display needle.

7. The device of claim 1, wherein the reservoir is formed of flexible material, wherein the reservoir changes shape responsive to the volume of medicament therein, and wherein the level indicator is responsive to the change in shape of the reservoir to indicate the medicament level.

8. The device of claim 7, further comprising a follower that follows the changes in shape of the reservoir and a linkage the couples the follower to the level indicator.

9. The device of claim 8, wherein the level indicator includes a mechanical indicator and wherein the linkage couples the follower to the mechanical indicator.

10. The device of claim 1, further comprising a housing covering the base and enclosing the reservoir and the pump, and wherein the housing includes a window through which the level indicator is viewable.

11. The device of claim 10, wherein the level indicator includes a display needle.
